(19) United States
(12) Patent Application Publication
(10) Pub. No.: US 2019/0019574 A1
(43) Pub. Date: Jan. 17, 2019

(54) SYSTEM AND METHOD FOR CREATING AN ELECTRONIC CONSENT-BASED MEDICAL RECORD

(71) Applicant: TruConsent, Dallas, TX (US)

(72) Inventor: Michael Byrnes, Frisco, TX (US)

(21) Appl. No.: 16/035,632

(22) Filed: Jul. 14, 2018

Related U.S. Application Data
(60) Provisional application No. 62/532,816, filed on Jul. 14, 2017.

(51) Int. Cl. G16H 10/60; G16H 70/20

(52) U.S. Cl. CPC G16H 10/60; G16H 80/00; G16H 70/20

(57) ABSTRACT

An electronic consent-based medical record is created by: providing a processor(s) communicably coupled to an input/output interface, a memory and a data storage; receiving a medical procedure identifier for a patient; creating the electronic medical record and storing the electronic medical record in the data storage; storing a medical data and an education information for a medical procedure associated with the medical procedure identifier in the electronic medical record; storing a set of consent-based questions for the medical procedure in the electronic medical record; providing the educational information and the consent-based questions for the medical procedure to the patient via a remote device; receiving an electronic consent from the patient; storing the electronic consent in the electronic medical record; providing a report based on the electronic medical record to a medical provider; receiving an electronic authorization from the medical provider; and storing the electronic authorization in the electronic medical record.

### PRIORITY CLAIM AND CROSS-REFERENCE TO RELATED APPLICATIONS

This patent application claims priority to U.S. Provisional Patent Application Ser. No. 62/532,816 filed on Jul. 14, 2017, the entire contents of which are incorporated herein by reference.

### FIELD OF THE INVENTION

The present invention relates in general to computer processing and data storage. In particular, the present invention relates to the creation and storage of electronic consent-based medical data.

### BACKGROUND OF THE INVENTION

Without limiting the scope of the invention, its background is described in connection with electronic medical data systems.

Patient-centered healthcare is actively promoted by today's healthcare industry. In fact, various agencies that govern the quality of U.S. health care have defined patient-centered care as “care that is respectful of and responsive to individual patient preferences, needs, and values” and that ensures “that patient values guide all clinical decisions.”

This definition shows that the most important part of patient-centered care is the involvement of patients when important health care decisions are needed, when patients have numerous options, and each option has different and important consequences with lasting outcomes. Examples include decisions about major surgery, chronic care medications that must be taken for the rest of one's life, the impact that opioid medication can have, and screening/diagnostic tests that can result in numerous grim and traumatic treatment options.

Yet, as medical science progresses and new options are being introduced that, although often improving outcomes, have inadvertently distanced physicians from their patients. Combine this with the time now required to meet federal and state mandates, this has resulted in a health care environment in which patients and their families are left out of important discussions, left feeling in the dark about how their problems are being managed, and how to navigate the overwhelming array of diagnostic and treatment options available to them. This lack of information results in patients making healthcare decisions without vital information, meaning most informed consents today-are not informed and are insufficient, suspect, and legally leave healthcare practitioners open to lawsuits for improper or no consent at all.

### SUMMARY OF THE INVENTION

Various embodiments of the present invention strive to put the patient at the center of health care decisions by providing the patient with healthcare information and/or healthcare options so that the patient can truthfully make an informed healthcare decision. Shared Decision-Making, as will be described in more detail below, increases patient literacy, promotes patient education, and delivers true physician-patient communication resulting in the patient being in control of their own healthcare decisions.

Shared Decision-Making is a process by which the optimal decision may be reached for a patient at a critical health crossroads involving, at minimum, a clinician and the patient, although other members of the health care team or friends and family members may be invited to participate. Moreover, in Shared Decision-Making, both parties share information: the clinician offers options and describes their risks and benefits, and the patient expresses his or her preferences and values.

For some decisions, there is one clearly superior path, and patient preferences play little or no role: a broken bone needs repair, advanced appendicitis equals surgery, and a bacterial infection requires antibiotics. For most medical decisions, however, more than one reasonable path forward exists (including the option of doing nothing, when appropriate), and different paths entail different combinations of possible therapeutic effects and side effects.

When more than one viable treatment or screening option exists, clinicians can facilitate Shared Decision-Making by encouraging patients to let clinicians know what they care about. The invention here can efficiently help patients absorb relevant clinical evidence and aid them in developing and communicating informed preferences, particularly for possible outcomes that they have not yet experienced.

The invention discussed here, engages both parties to share information. The clinician offers options and describes their risks and benefits, the patient expresses his or her beliefs, preferences, and values thru interaction/participation with the application. Each participant is thus armed with a better understanding of the relevant factors and shares responsibility in the decision about how to proceed. This patient centered-Shared Decision-Making interaction is fostered by the invention and will now offer patients the ability to sign a TRUE INFORMED CONSENT.

A platform is provided to facilitate Shared Decision-Making. This acknowledges the inefficient treatment decision model of one-way communication. It resolves the conflict between autonomy, health, and the variance between the values of the patient and the values of the physician. A unique educational experience is provided that addresses the need for patients to accept responsibility and have a greater role in their own healthcare. There are very few applications or programs available for healthcare practices to facilitate decision making. The existing market does not offer an integrated shared decision-making, educational and consent delivery model. Thus, the methods and platforms described herein afford today's patients a mechanism to make better health care decisions they feel would work best for their own personal needs and beliefs.

In one embodiment, a computerized method for creating an electronic consent-based medical record comprises: providing one or more processors communicably coupled to an input/output interface, a memory and a data storage; receiving a medical procedure identifier for a patient via the input/output interface; creating the electronic consent-based medical record for the patient and storing the electronic consent-based medical record in the data storage using the one or more processors; storing a medical data and an education information for a medical procedure associated with the medical procedure identifier in the electronic consent-based medical record using the one or more processors; storing a set of consent-based questions for the medical procedure for the patient in the electronic consent-based medical record using the one or more processors; providing the educational information and the consent-based questions for the medical procedure to the patient via a remote device communicably coupled to the input/output interface using the one or more processors; receiving an electronic consent from the patient via the input/output interface; storing the electronic consent in the electronic consent-based medical record using the one or more processors; providing a report based on the electronic consent-based medical record to a medical provider via the input/output interface; receiving an electronic authorization from the medical provider via the input/output interface; and storing the electronic authorization in the electronic consent-based medical record using the one or more processors.

In one aspect, the method further comprises obtaining the medical data for the patient, the educational information for the medical procedure or a combination thereof. In another aspect, the method further comprises obtaining or generating the set of consent-based questions for the medical procedure associated with the medical procedure identifier for the patient. In another aspect, the method further comprises creating an executable package or file using the one or more processors that provides the education information or the consent-based questions or both to the patient when activated by the remote device, and wherein providing the educational information and the consent-based questions comprises sending the executable package or file to the remote device via the input/output interface. In another aspect, the consent-based questions comprise an automated question-answer session. In another aspect, the method further comprises requesting the electronic consent from the patient after a successful completion of the consent-based questions. In another aspect, the electronic consent comprises an electronic signature, a biometric identifier, an image of the patient taken by the remote device, or a combination thereof. In another aspect, the method further comprises receiving a request for an additional information regarding the medical procedure from the remote device; and providing the additional information to the remote device or notifying the medical provider of the request. In another aspect, the method further comprises generating the report based on the electronic consent-based medical record. In another aspect, the method further comprises updating a patient health record based on the electronic consent-based medical record. In another aspect, the method further comprises generating a bill or medical reimbursement request based on the electronic consent-based medical record. In another aspect, the method further comprises providing the educational information and/or the consent-based questions to the medical provider prior to providing the educational information and the consent-based questions to the patient; receiving an authorization to provide the consent-based question to the patient; and storing the authorization in the electronic consent-based medical record. In another aspect, the method further comprises providing a patient report based on the electronic consent-based electronic record. In another aspect, the educational information comprises one or more of a video, an audio recording, an electronic document, an electronic presentation, an image, or a hyperlink. In another aspect, all communications via the input/output interface are encrypted.

In another embodiment, a system for creating an electronic consent-based medical record comprises: an input/output interface; a memory; a data storage; a remote device communicably coupled to the input/output interface; one or more processors communicably coupled to the input/output interface, the memory and the data storage, wherein the one or more processors receive a medical procedure identifier for a patient via the input/output interface, create the electronic consent-based medical record for the patient and store the electronic consent-based medical record in the data storage, store a medical data and an education information for a medical procedure associated with the medical procedure identifier in the electronic consent-based medical record, store a set of consent-based questions for the medical procedure for the patient in the electronic consent-based medical record, provide the educational information and the consent-based questions for the medical procedure to the patient via the remote device, receive an electronic consent from the patient via the input/output interface, store the electronic consent in the electronic consent-based medical record, provide a report based on the electronic consent-based medical record to a medical provider via the input/output interface, receive an electronic authorization from the medical provider via the input/output interface, and store the electronic authorization in the electronic consent-based medical record.

Note that the foregoing method and any of the aspects can be implemented using a non-transitory computer readable medium that when executed by the one or more processors performs the stated functionality.

In on aspect, the one or more processors obtain the medical data for the patient, the educational information for the medical procedure or a combination thereof. In another aspect, the one or more processors obtain or generate the set of consent-based questions for the medical procedure associated with the medical procedure identifier for the patient. In another aspect, the one or more processors create an executable package or file that provides the education information or the consent-based questions or both to the patient when activated by the remote device, and wherein providing the educational information and the consent-based questions comprises sending the executable package or file to the remote device via the input/output interface. In another aspect, the consent-based questions comprise an automated question-answer session. In another aspect, the one or more processors request the electronic consent from the patient after a successful completion of the consent-based questions. In another aspect, the electronic consent comprises an electronic signature, a biometric identifier, an image of the patient taken by the remote device, or a combination thereof. In another aspect, the one or more processors: receive a request for an additional information regarding the medical procedure from the remote device; and provide the additional information to the remote device or notifying the medical provider of the request. In another aspect, the one or more processors generate the report based on the electronic consent-based medical record. In another aspect, the one or more processors update a patient health record based on the electronic consent-based medical record. In another aspect, the one or more processors generate a bill or medical reimbursement request based on the electronic consent-based medical record. In another aspect, the one or more processors: provide the educational information and/or the consent-based questions to the medical provider prior to providing the educational information and the consent-based questions to the patient; receive an authorization to provide the consent-based question to the patient; and store the authorization in the electronic consent-based medical record. In another aspect, the one or more processors provide a patient report based on the electronic consent-based electronic record. In another aspect, the educational information comprises one or more of a video, an audio recording, an electronic document, an electronic presentation, an image, or a hyperlink. In another aspect, all communications via the input/output interface are encrypted.

The foregoing is a summary and thus contains, by necessity, simplifications, generalizations, and omissions of detail. Consequently, those skilled in the art will appreciate that this summary is illustrative only and is not intended to be in any way limiting. Various other aspects, features and advantages of data creation, storage, management and processing are set forth in the teachings of the present disclosure, such as the claims, text, and drawings set forth herein.

### BRIEF DESCRIPTION OF THE DRAWINGS

For a more complete understanding of the features and advantages of the present invention, reference is now made to the detailed description of the invention along with the accompanying figures, in which:

FIG. 1 depicts a pictorial representation of a Shared Decision-Making integrated application operating in a network of data processing systems in which illustrative embodiments of the present invention may be implemented;

FIG. 2 is a functional block diagram of an example Shared Decision Making patient data request of callup, a Share Decision-Making modality preparation, and the publication of the Shared Decision-Making modality to physician tablet computers in accordance with one embodiment of the present invention;

FIG. 3 illustrates a process flow in accordance with one embodiment of the present invention;

FIG. 4 sets forth the TruConsent Shared Decision-Making outsourcing system patient appointment arrival Tablet Loading process in accordance with one embodiment of the present invention;

FIG. 5 depicts a sample patient flow in accordance with one embodiment of the present invention;

FIG. 6 depicts a data flow in accordance with one embodiment of the present invention;

FIG. 7 depicts an information flow in accordance with one embodiment of the present invention;

FIG. 8 depicts an education system data-process flow in accordance with one embodiment of the present invention;

FIG. 9 depicts an outsourcing shared decision making, review and data movement flow (backside) in accordance with one embodiment of the present invention;

FIG. 10 depicts a process flow in accordance with one embodiment of the present invention;

FIGS. 11A-11D are screen shots of yes/no questions presented on a device to a patient for a MRI scan in accordance with one embodiment of the present invention;

FIG. 12 is a screen shot of yes/no questions presented on a device to a patient for a pain medication in accordance with one embodiment of the present invention;

FIG. 13 depicts a system for creating an electronic consent-based medical record in accordance with one embodiment of the present invention; and

FIGS. 14A-B depict a computerized method for creating an electronic consent-based medical record in accordance with one embodiment of the present invention.

### DETAILED DESCRIPTION OF THE INVENTION

Illustrative embodiments of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure

Various embodiments of the present invention strive to put the patient at the center of health care decisions by providing the patient with healthcare information and/or healthcare options so that the patient can truthfully make an informed healthcare decision. Shared Decision-Making, as will be described in more detail below, increases patient literacy, promotes patient education, and delivers true physician-patient communication resulting in the patient being in control of their own healthcare decisions.

Shared Decision-Making is a process by which the optimal decision may be reached for a patient at a critical health crossroads involving, at minimum, a clinician and the patient, although other members of the health care team or friends and family members may be invited to participate. Moreover, in Shared Decision-Making, both parties share information: the clinician offers options and describes their risks and benefits, and the patient expresses his or her preferences and values.

For some decisions, there is one clearly superior path, and patient preferences play little or no role: a broken bone needs repair, advanced appendicitis equals surgery, and a bacterial infection requires antibiotics. For most medical decisions, however, more than one reasonable path forward exists (including the option of doing nothing, when appropriate), and different paths entail different combinations of possible therapeutic effects and side effects.

When more than one viable treatment or screening option exists, clinicians can facilitate Shared Decision-Making by encouraging patients to let clinicians know what they care about. The invention here can efficiently help patients absorb relevant clinical evidence and aid them in developing and communicating informed preferences, particularly for possible outcomes that they have not yet experienced.

The invention discussed here, engages both parties to share information. The clinician offers options and describes their risks and benefits, the patient expresses his or her beliefs, preferences, and values thru interaction/participation with the application. Each participant is thus armed with a better understanding of the relevant factors and shares responsibility in the decision about how to proceed. This patient centered-Shared Decision-Making interaction is fostered by the invention and will now offer patients the ability to sign a TRUE INFORMED CONSENT.

A platform is provided to facilitate Shared Decision-Making. This acknowledges the inefficient treatment decision model of one-way communication. It resolves the conflict between autonomy, health, and the variance between the values of the patient and the values of the physician. A unique educational experience is provided that addresses the need for patients to accept responsibility and have a greater role in their own healthcare. There are very few applications or programs available for healthcare practices to facilitate decision making. The existing market does not offer an integrated shared decision-making, educational and consent delivery model. Thus, the methods and platforms described herein afford today's patients a mechanism to make better health care decisions they feel would work best for their own personal needs and beliefs

The Shared Decision-Making application described herein provides an outsourced solution to foster both patient, and physician decision making regarding medical diseases and conditions. The integrated application includes numerous components that can coexist in a health care data processing environment. These healthcare components can be hardware components, software components, or a combination thereof. For example, any number of physician tablets, practice data storage devices, networking equipment, server applications, insurance billing applications, databases, client applications, virtual servers, logical partitions, and partition management firmware can be found in a typical data processing environment.

The Outsource Service of Shared Decision-Making operates in the data processing environment can depend upon any number of other components in the data processing situation for providing their intended functionalities. For example, a physician's application cannot function if the tablet hardware executing the physician application crashes. As another example, the physicians' tablet/application may receive a timeout or failure notification if a web-server application executing on a remote server computer cannot be reached, either because the web-server application is busy, the remote server computer is experiencing an error, or a network link between the two computers is down. Moreover, the TruConsent invention/application executing in an application server depend on a database managed by a database management application executing in another server.

Complex data processing environments can include thousands if not millions of hardware, firmware, and software components. Consequently, a large number of relationships can exist between the components in such an environment. Furthermore, not all relationships are the same. For example, in one case, a component can continue to function if a related component is unavailable or delayed. In another case, a component may experience a catastrophic failure if a related component goes offline.

In one example, the application is designed to automatically integrate into the HL7's Version 2.x (V2) messaging standard. This standard is the workhorse of electronic data exchange in the clinical domain and arguably the most widely implemented standard for healthcare in the world. The application/platform has extendible standards that permit structured, coded healthcare information of the type required to support patient care, to be exchanged between physician computer applications, and the application, while preserving the meaning. Further, this application/platform partners with healthcare information technology users (physicians) to ensure that HL7 standards meet real-world requirements and appropriate standards. The application/platform is flexible and has built-in seamless development options that once initiated, will meet emergent requirements in the healthcare data integration management.

The application/platform communicates with many combinations of EHR-systems and archives in use today. The application/platform is designed to exploit the principal repository of healthcare data and treatment information (the EHR system). EHR systems contain a patient's personal details (including name, address, and date of birth), a summary of the patient's medical history, and documentation of each event, including symptoms, diagnosis, treatments, and outcome. Relevant documents and correspondence are also included. Each health care provider involved in or contributing to a patient's care maintains documentation regarding patient treatment and interaction. The primary purpose of the medical record is to act as a communication tool among healthcare providers.

The application/platform takes primary identified patient data, that once queried, the application triggers the integrated search of the Physicians EHR system. Once this data is located, the information needed for the Shared Decision-Making visit is exported from the physician's EHR system as standardized EHR extracts to a tablet or computer. For each medical/surgical procedure, the invention data exported exports a video of the medical/surgical procedures, a peer reviewed unique informed consent, patient information handouts, and any additional pertinent treatment related materials. This material is then compiled, checked against the EHR record, and transmitted to a tablet where the patient can engage the Shared Decision-Making process.

A patient should be educated to truly understand and sign a legally sufficient informed consent form. Shared Decision-Making is the process that healthcare has focused on to support patients in their decision making. Various embodiments of the present invention solve the Shared Decision-Making problem by integrating the education (video, handout, web-resources) with a re-designed, peer-reviewed informed consent, and adds the independent physician review and summary report of the entire Shared Decision-Making process. In one embodiment, the Shared Decision-Making application/platform is a software program that is inside the TruConsent computer network that provides an outsourced Shared Decision-Making solution. The computer network has a server that is in communication with numerous physician-client computers, tablets or other smart-tech devices. The physician client computers reside in their respective offices, clinics, and hospitals. The TruConsent server facilitates the command and control of a library of medical procedure/treatment information and the distribution of information to client computers upon request.

In addition to the server storing the library of information, the server is also the site where patient photos are maintained. A picture of each patient signing their individual informed consent form is made documenting the patient's completion of the Shared Decision-Making process. This information is stored in the server and inside the Physician/Patient's (EHR) file documenting the patient consent.

Any discussions between patients and physicians about any relevant procedure related issues, is documented in the application as part of the Shared Decision-Making process and stored in the server for each individual patient.

The information presented to the patient is in a format that is understandable and drafted between the 5th and 6th grade reading level(s). Prior consent systems are mass produced, and written assuming a level of education or comprehension. The education level of the population of patients is not the same, which leaves most patients feeling uninformed, too embarrassed to say they do not understand, and signing the consent form even though they don't recognize the issues relative to the procedure.

Sadly, most physicians are unable to communicate with a patient on an equal footing. Shared decision making is the mechanism to level the communication paradigm. The application/platform engages both parties to share information: the clinician offers options and describes their risks and benefits, and the patient expresses his or her beliefs, preferences, and values thru the application. Each participant is thus armed with a better understanding of the relevant factors and shares responsibility in the decision about how to proceed. The patient now has the tools to sign a TRUE INFORMED CONSENT.

One non-limiting embodiment of the present invention includes the following features:
1. Patient Data Query is entered into a tablet or other device;
2. Patient Data/Physician EMR Adapter-Interface accesses the physicians EMR DATA to engage the Semantic Annotation System, triggering the Decision Support Interface directing the TruC-process search engine, accessing the Consent Library, seeking Informed Consent form;
3. Search the corresponding video searching the Medical Procedure Video;
4. Engage the Medical Procedure Video Rule Engine assigning the video to prepare to load;
5. Attach correct video to the Informed Consent creating a completed modality;
6. Verify Modality is correct;
7. Invoke/load the correct Modality (consent and video) to populate the data fields and inserted into the tablet.
8. Correct SDM Modality is the loaded for the patient to engage with;
9. After the Video and other educational materials are reviewed, the questions are answered (YES/NO);
10. Questions answered “NO”, are checked in the Decision Support Interface the “NO” answers are flagged for follow-up;
11. Additional Modality data interface is accessed with ------- the “NO” question being flagged in a (File for Physician/Staff follow-up), and the additional materials for the “NO” answers directed back to the patient, directing the patient to re-answer the “NO” questions determining if the patient can now answer “YES” that he/she understands;
12. Patient Finishes questions (EITHER all are answered “YES”, or patient needs to have additional information provided by staff/physician in order to answer “YES”);
13. ONLY upon all answers are a “YES”, will the signature field be accessed and the patient is prompted to sign their name;
14. As the patient's picture is taken and incorporated into the report and directed to turn in the tablet to the staff;
15. Staff/Physician sign the module confirming that the patient completed the SDM modality;
16. Completed modality is sent through the Integration APP to the SDM-Decision Support Interface;
17. A completed copy of the modality is sent to the patients (EMR/EHR) at the physician's office;
18. From the Decision Support Interface a copy of the completed modality is encrypted and sent to the TRUC Physician to review the SDM modality completed by the patient and verify the completeness of the modality;
19. The completed physician SDM report is sent through the Integration APP to the Decision Support Interface; and
20. A copy of the Report is then routed to the Physician EMR for the patients file.

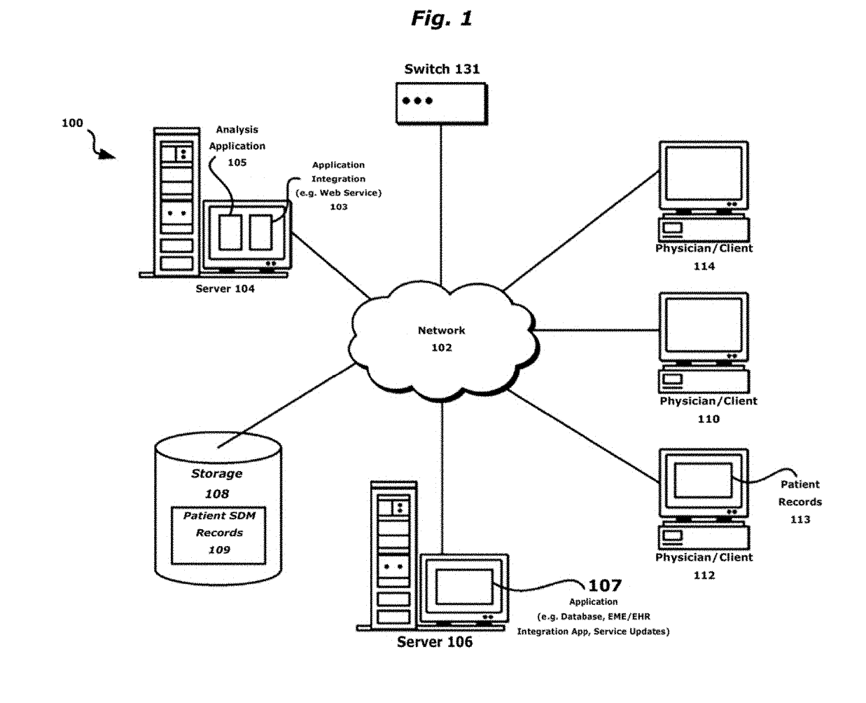

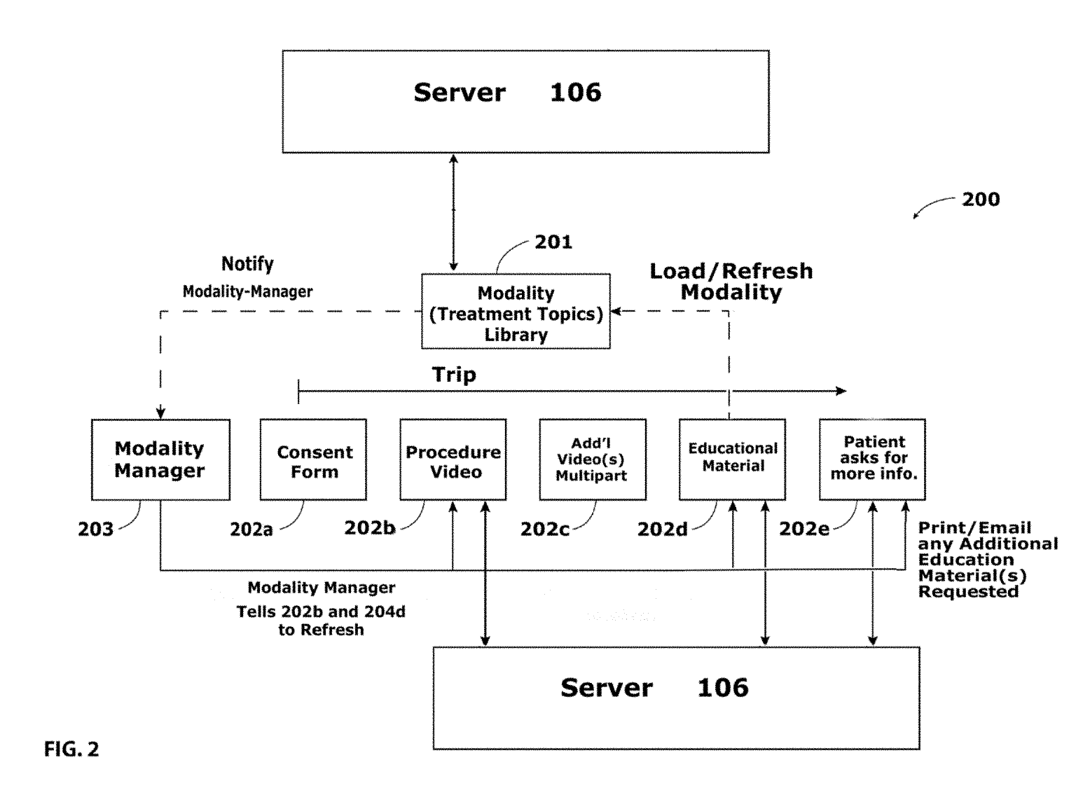

Any advantages listed herein are only examples and are not intended to be limiting to any aspect of the invention listed here. Additional or different uses may be realized. Furthermore, an illustrative embodiment may have some, all, or none of the advantages listed above. With reference to the figures and with reference to FIGS. 1 and 2, these figures are example diagrams of data processing environments in which illustrative embodiments may be implemented. FIGS. 1 and 2 are only examples and are not intended to assert or imply any limitation with regard to the environments in which different embodiments may be implemented. An implementation may make many modifications to the depicted environments based on the following description.

FIG. 1 depicts a pictorial representation of a Shared Decision-Making integrated application operating in a network of data processing systems in which illustrative embodiments may be implemented. Data processing environment 100 is a network of computers in which the illustrative embodiments may be implemented. Data processing environment 100 includes network 102. Network 102 is the medium used to provide communications links between various devices and computers connected together within data processing environment 100. Network 102 may include connections, such as wire, wireless communication links, or fiber optic cables. Server 104 and server 106 are communicably coupled to network 102 along with storage unit 108. Software applications may execute on any computer in data processing environment 100. In addition, clients 110, 112, and 114 are communicably coupled to network 102. A data processing system, such as server 104 or 106, or client 110, 112, or 114, may contain data and may have software applications or software tools executing thereon. Only as an example, and without implying any limitation to such architecture, FIG. 1 depicts certain components that are usable in an example implementation of an embodiment.

For example, switch 131 is an example networking equipment component, of which there can be any number present in a given implementation. Analysis application 105 in server 104 is an implementation of an embodiment described herein. In an example operation, application 105 identifies the relationships of application 103, which for example may be an EMR/HER integration application web server application executing in server 104.

For example, analysis application 105 analyzes patient EMR/EHR records 109 in storage 108, which may be system or event-generated, log records 113 in client 112, which may be user-provided, to determine the relationships in which application 103 participates. By performing one or more operations described herein, analysis application 105 may find that application 103 is related to application 107, which may be a database or a web service, storage 108, and switch 131. Servers 104 and 106, storage unit 108, and clients 110, 112, and 114 may couple to network 102 using wired connections, wireless communication protocols, or other suitable data connectivity.

Clients 110, 112, and 114 may be, for example, tablet computers or network computers. In the depicted example, server 104 may provide data, such as booting information, operating system images, files related to the operating system and other software applications, and applications to clients 110, 112, and 114. Clients 110, 112, and 114 may be clients to server 104 in this example. Clients 110, 112, 114, or some combination thereof, may include their own data, booting instructions, operating system images, files related to the operating system and other software applications. Data processing environment 100 may include additional servers, clients, and other devices that are not shown. In the depicted example, data processing environment 100 may be the Internet.

Network 102 may include connections, such as wire, wireless communication links, or fiber optic cables. Server 104 and server 106 are communicably coupled to network 102 along with storage unit 108. Software applications may execute on any computer in data processing environment 100. In addition, clients 110, 112, and 114 are communicably coupled to network 102. A data processing system, such as server 104 or 106, or client 110, 112, or 114, may contain data and may have software applications or software tools executing thereon. Only as an example, and without implying any limitation to such architecture, FIG. 1 depicts certain components that are usable in an example implementation of an embodiment. For example, switch 131 is an example networking equipment component, of which there can be any number present in a given implementation.

Analysis application 105 in server 104 is an implementation of an embodiment described herein. In an example operation, application 105 identifies the relationships of application 103, which for example may be a web server application executing in server 104. For example, analysis application 105 analyzes log records 109 in storage 108, which may be system or event-generated, log records 113 in client 112, which may be user-provided, to determine the relationships in which application 103 participates.

By performing one or more operations described herein, analysis application 105 may discover that application 103 is related to application 107, which may be a database or a web service, storage 108, and switch 131. Servers 104 and 106, storage unit 108, and clients 110, 112, and 114 may couple to network 102 using wired connections, wireless communication protocols, or other suitable data connectivity.

Clients 110, 112, and 114 may be, for example, tablets, personal computers or network computers. In the depicted example, server 104 may provide data, software applications, and applications to clients 110, 112, and 114. Clients 110, 112, and 114 may be clients to server 104 in this example. Clients 110, 112, 114, or some combination thereof, may include their own data, boot files, operating system images, files related to the operating system and other software applications. Data processing environment 100 may include additional servers, clients, and other devices that are not shown. In the depicted example, data processing environment 100 may be the Internet.

Network 102 may represent a collection of networks and gateways that use the Transmission Control Protocol/Internet Protocol (TCP/IP) and other protocols to communicate with one another. Of course, data processing environment 100 also may be implemented in numerous types of networks, such as for example, an intranet, a local-area network (LAN), or a wide area network (WAN) and all transmissions would be encrypted to meet patient privacy rights and governmental regulations. FIG. 1 is intended as an example, and not as an architectural limitation for the different illustrative embodiments.

FIG. 2 is a functional block diagram of an example Shared Decision Making patient data request of callup, a Share Decision-Making modality preparation, and the publication of the Shared Decision-Making modality to physician tablet computers identified as 110, 112, 114 for a patient visit. The Shared Decision-Making Modality Treatment Topics Library 201 accepts the patient inquiry from server 106 and accesses the patient (EHR/EMR) database, which triggers the Shared Decision-Making Modality Manager 203.

The Shared Decision-Making Modality Manager 203 which identifies the data topic (modality to be discussed) and loads the modality specific data in each of the folders 202a-202e, and can instruct any one or more of the folders to take further action. In this example, the loading of the modality to be published by folder 202d requires that the folders 202b and 202d refresh one or more objects being displayed on the physician's tablet device 110, 112, 114.

To publish a Shared Decision-Making modality identified by model 200 showing five folders 202a-202e in this example, the folders are accessed internally in sequence in the server and once prepared then sent to the physician's tablet computer(s) 110, 112, 114. Each of the folders 202a-202e is accessed in a temporal sequence, which is referred to herein as a trip. Thus, folder 202a was the first folder to be accessed, which would trigger folder 202b and so on. One of the folders, in this example the folder 202d, publishes a topic which refresh folders and be noted in the Topics Manager 201.

The Shared Decision-Making Modality Manager 203 instructs the folders 202b and 202d to refresh, and the folders 202b and 202d call a server 106, to refresh. Any data manipulation or calculations required to update objects associated with the folders 202b, 202d are carried out by the server 106. For example, if an object associated with the folders 202b, 202d needs to be updated on the display device 112, the server 106 will return the value(s) or other parameters required for the folders 202b, 202d to refresh on the display device 112. No other folders are refreshed, which means that folders 202a, 202c, and 202e are not refreshed while folders 202b and 202d are being refreshed.

Moreover, the number of folders required to be tracked by the various computing devices in the system (chart 100) is dramatically reduced, allowing for a seamless and real-time flow and updated modality loading on numerous physician computers (chart 110, 112, 114) simultaneously.

Before describing in detail the improved system architecture and application for the outsourcing Shared Decision-Making interaction, various embodiments of the present invention provide novel data structures of the system software and interactions with users, as opposed to combinations of established system devices. Examples of a system apparatus are a computer, database, telephone network, PBX system or a communication system linking the system apparatus by a local area network, wide area network, or Internet network.

Various embodiments of the present invention utilize discrete sub systems or sub-assembly components, and associated control of the system mentioned apparatus and components through the application. Command, control, the data construction, and arrangement of the present invention have been illustrated in the drawings by readily understandable block diagrams. Because the structural details prevalent in modern computer component design, the drawings show only those specific details that are relevant to the invention presented here.

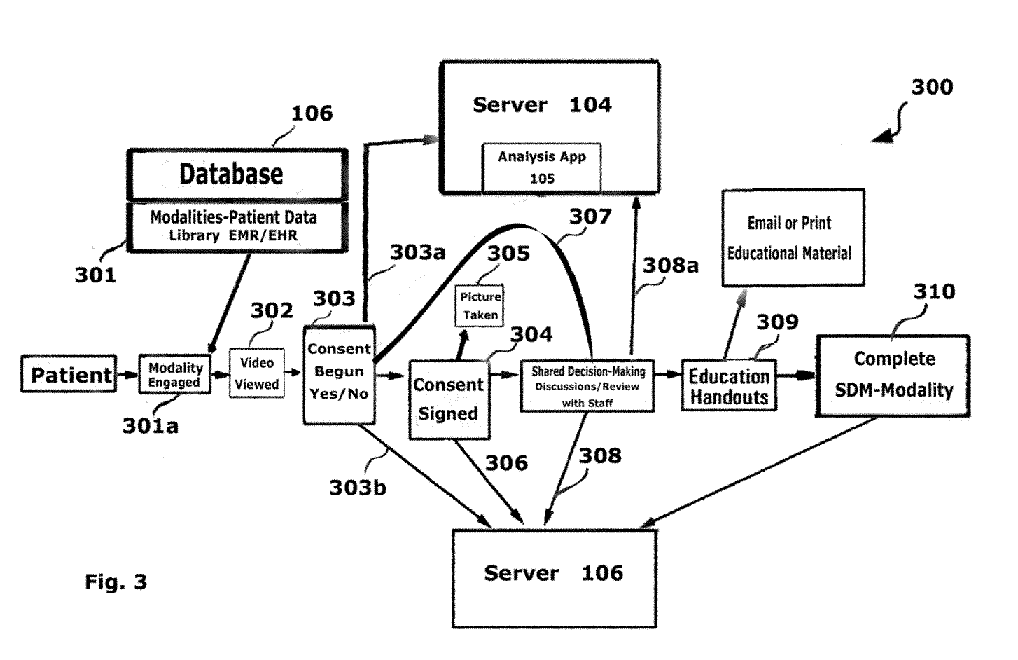

FIG. 3 illustrates a process flow in accordance with one embodiment of the present invention. A software program 300 resides in the memory of a computer network. The computer network has a server that is in communication with numerous client computers. The client computers reside in doctors' offices, clinics, hospitals, and personal electronic devices. The server facilitates the command and control of a library or archive 106, 301 of medical/surgical procedure information and the distribution of information to client computers upon request.

For each modality 301a, there is an informed consent form 304, educational video 302, and patient education materials 308. For medical/surgical procedures, the server 106 has an informed consent form, an educational video attached to it, and patient information handouts.

In addition to storing the library of information, the server is also the site where patient pictures are maintained. A photo of each person signing the informed consent form is made documenting the person reading and signing the written consent. This information is stored to document the patient consenting to the procedure and the information that was received by the patient. This picture documents the patients' acknowledgement of the benefits, risks, and options of the medical/surgical procedure, that the patient received this information on a specific date and time. With informed consents of patients stored electronically, the consent can be accessed in the future to verify the patients level of acceptance, and document that the patient had participated in the Shared Decision-Making process.

Additionally, this outsourced Shared Decision-Making invention protects the physician (by reducing risks) from legal challenges because the stored data will document what was the patient educated about, did he/she acknowledge understand and accept the risks, and therefore he/she in fact had knowledge of these facts before the procedure was initiated. This will reduce the instances of patients claiming that they did not know or were not told of the risks associated with the Shared Decision-Making informed consent process.

The Shared Decision-Making process as detailed in FIG. 3 is a result of client staff entering patient name into the invention/application tablet computer. This accesses the database 106, triggering the EMR/EHR integration accessing the client's patients files 301, loading the modality for the patient to complete 301a.

The patient then begins the program by viewing the video 302, after the video ends the patient is directed to special designed consent forms (YES/NO) 303. The patient reads/answers each fact pattern and answers questions 303b, and if there are any “NO” answers 303a, at the end of the consent the patient is directed back to a NO answer and provided additional information. Should the answer remain NO, the question is called out for staff/patient review 307. Only upon a YES to all answers can the consent be signed 304. Upon patient signing the consent, the patients picture is taken 305, and the signature and photo are stored on the server 106.

After the patient has signed 304 and the photo being taken 305, the patient is directed to turn the tablet into staff where the staff and patient review and discuss the procedure, alternatives, patient's beliefs, and goals 308, and all NO answers are reviewed by staff 308a. Upon completion the staff/patient discussion, the staff signs the form and the data is stored upon server 106. Any educational handouts are handed to the patient or emailed 309, and once the modality has been completed 310 the report is stored in server 106.

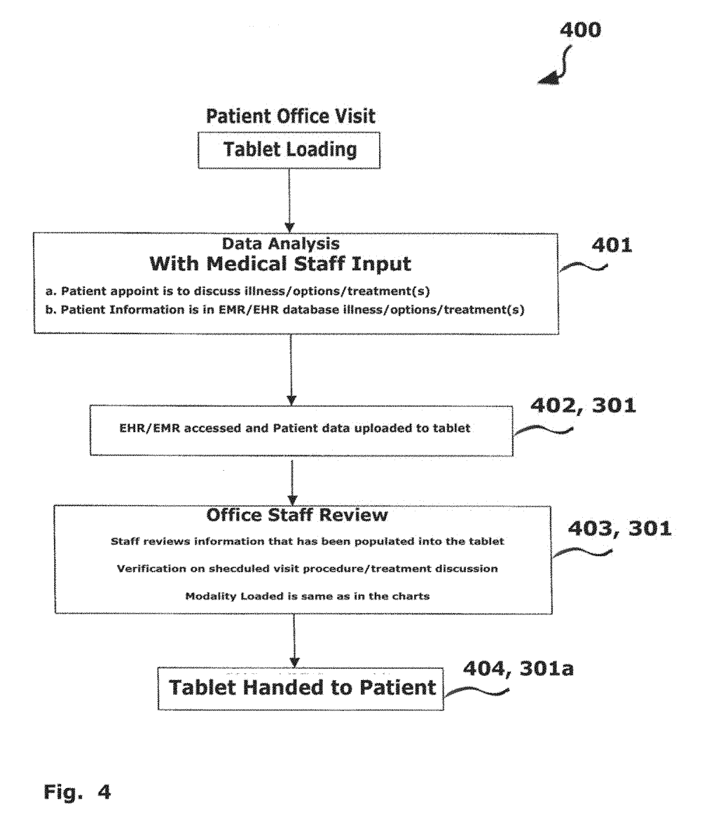

FIG. 4 sets forth the TruConsent Shared Decision-Making outsourcing system patient appointment arrival Tablet Loading process in general 400. This development process is repeated for all clients, 110, 112, 114 with the only variations needed to accommodate different modalities, treatments, diseases, and their respective treatments.

When a patient arrives for their appointment 401, they will be handed a tablet 404, 301a, with their personal information, modality, and Shared Decision-Making data loaded. The tablet loading process of combining the physician's EMR/EHR systems with the invention distinguishes the Shared Decision-Making application invention from all other computer aids in medicine.

The application is accessed by the staff entering a patients' basic information (NAME, SSN, etc.) into the tablet of computer system 401. The application accesses the physician's EMR/EHR records through the inventions integration program 402, 301. The application auto-populates the modality information with the patient's information including the proposed treatment, or area of illness—treatment the patient is scheduled to discuss 201. The staff then confirms the loaded information is for the correct patient 403,301 and the tablet or computer is handed to the patient for their review and completion 404, 301a.

Individually, various embodiments of the present invention provide the outsourcing of Shared Decision-Making that includes educational materials (including the video), which are used to support patient literacy and provide a basis to answer the consent including a patient who may wish to withhold consenting and choosing another avenue of treatment. This involves peer reviewed consent forms that are drafted at a 5th to 6th grade reading level to assist in the comprehension of complex medical terminology and methods. This allows a uniform consent that can be tailored to each physician's practice. Additionally, the consent form delivers the treatment options and the “NO” treatment options and anticipated results for each choice.

Various embodiments of the present invention solve the shared decision-making problem by integrating the education (video, handout, web-resources) with a re-designed peer-reviewed informed consent, and adds the independent physician review and report of the entire SDM process. The present invention can be a software program that is inside the TruConsent computer network that provides an outsourced shared decision-making solution. The computer network has a server that is in communication with numerous physician-client computers, tablets or other smart-tech devices.

The physician client computers reside in their respective offices, clinics, and hospitals. The TruConsent server facilitates the command and control of a library of medical procedure/treatment information and the distribution of information to client computers upon request.

For each medical/surgical procedure there is a video, peer reviewed consent, and patient educational materials. For each procedure, the server compiles the modality consisting of a video of the medical/surgical procedures, the peer reviewed unique informed consent, patient information handouts, and any additional pertinent treatment related materials. This material is then transmitted to a tablet where the patient can engage the SDM process.

In addition to the server storing the library of information, the server is also the site where patient photos are maintained. A picture of each patient signing their individual informed consent form is made documenting the patient's completion of the SDM process. This information is stored forever in the TruConsent server and inside the physician/Patient's (EHR) file documenting the patient consent.

The discussion between the patient and the physician, about any relevant procedure related issues is documented in the TruConsent APP as part of the Shared Decision-Making process and stored in the TruConsent server for each individual patient. The information presented to the patient is in a format that is understandable and drafted between the 5th and 6th grade reading level(s). Prior consent systems are mass produced, and written assuming a level of education or comprehension. The education level of the population of patients is not the same, which leaves most patients feeling uninformed, too embarrassed to say they do not understand, and signing the consent form even though they don't recognize the issues relative to the procedure. Sadly, most physicians are unable to communicate with a patient on an equal footing. Shared Decision-Making is the mechanism to level the communication paradigm.

Through the combination of the diverse educational materials and the peer reviewed consent, the patient engagement is elevated, patient literacy is increased, and a true partnership is promoted between health provider and patient. This equal partnership is achieved through the promotion of literacy and facts provided to the patient to ask specific questions about his/her treatment plans, goals, and objectives. Moreover, the program highlights what doing nothing (No-treatment) and alternatives to a proposed treatment are available and the expected results for doing nothing.

Logic Gates:
1) IF patient data entered into tablet then load corresponding (consent);
2) IF consent loaded then load matching educational video;
3) If patient has an email address then copy of completed SDM is emailed to patient;
4) If patient checks box marked additional info requested then inquiry routed to treating physician and to TruConsent;
5) If additional info request is routed to TruConsent then materials relevant to the modality are compiled and emailed to patient;
6) If each question answered “YES” then signature box is engaged;
7) If any question is answered “NO” then that question is flagged for additional input;
8) If question is flagged then access data base for additional educational resources;
9) If question is flagged then question is called out for physician-staff to review;
10) If “NO” question is now answered “YES” the program looks for any additional “NO” answers;
11) If there are no “NO” answers then signature box is engaged;
12) If signature box engaged then Photo of patient is captured and stored;
13) If signature box is completed then verify the photo has been taken;
14) If there is no photo (Patient objects) then mark specific item in document addressing patient's refusal;
15) If the signature and photo have been completed then patient is directed to turn tablet into the staff,
16) If patient has handed tablet to Physician/staff then the “NO” answered questions are called out for review;
17) If satisfied with interaction and patients understanding of “NO” questions and the reasons for “YES” then the staff checks box indicating review of material with patient specifically listing all “NO” answers and verification that the materials were specifically addressed with patient;
18) If there are no more “NO” questions to address or if all answers were a “YES” then signature callout box is opened for staff signature;
19) If staff signature is completed then a review checking for completed questions, patient signature, patient photo and staff signature is verified complete;
20) If modality and signatures/phot is complete, then the report is sent to the patients' electronic health record (MHR/EHR);
21) If the completed modality report is sent to the patients EHR, then a copy is sent to TruConsent for independent physician review
22) If the independent physician review is completed and a report is generated then the completed SDM modality Report is combined with the independent physician report and sent to treating physician for the patients (EHR)

A non-limiting embodiment of the present invention involves the development of an integrated software application that resides on a network server. The computer network server is designed to be in communication with a multitude of physician-client computers, tablets or personal electronic devices. The client computers and tablets located where medical procedures are performed (doctors' offices, clinics, hospitals).

The server facilitates the application's command and control of a library or archive of medical consent forms for specific treatments and surgical procedures. This archive would also contain a library of educational videos, and trigger the distribution of information to client computers upon request.

Specialized written consent forms written to deliver treatment relevant information via a statement and a question in a manner to confirm the patients understanding are used. The APP/program/server would link background information to extracted concepts, access the library, and insert the matching educational video and links for additional procedure information. This modality would be loaded into the tablet and handed to the patient upon checking in to the office. For patients with no pre-visit access, the integrated application would communicate with the doctors EHR system and have a specific consent, modality, and treatment data loaded before the patient received the tablet.

In addition to storing the library of information, the server is also the site where patient photos are stored. A picture of each patient signing their informed consent is captured. This information is embedded into the patients record to document the patient consenting to the procedure, and the information that was received by the patient.

The application would include integration of the database allowing patients electronic access to treatment relevant information prior to their visits.

The application would also measure the time spent on each question and flag or callout any answer indicating the patient did not understand.

In one embodiment of the present invention, the following components/features are necessary:
1. The software Application;
2. The integration program for communication with a multitude of physician-client computers, tablets or personal electronic devices;
3. Program downloading, upon request, selected modality consisting of informed consent, video, and educational material to tablet computer operated device;
4. The APP's command and control of a library or archive of medical consent forms for specific treatments and surgical procedures.

The following component/features are optional:
1. Tracking time spent on each question;
2. Integration with EMR to send data to patient's email;
3. Integrated data sent to patient highlighting additional/optional/non-treatment options;
4. Link to Client-physician EMR and access appointment data before patient visit, to contact patient prior to visit and provide links to information about upcoming appointment;
5. Automatically follow up with patient post procedure to send post treatment/recovery reminders, additional diagnostic/treatment information;
6. Option to video entire physician patient encounter to document full Shared Decision-Making discussions (including options—and doing nothing);
7. Ability to access computer network for updated information on a patient and compare the patient info to said stored profile; updating patient profile;
8. Updating information and develop a new informed consent with patient info.

The elements can be reconfigured to allow:
1. Consenting for events/social occasions (attending college party) recognizing the inherent activities usually associated, and consenting;
2. Capture consent from a potential sexual partner (video) to show state of sobriety and freely signing;
3. Used in conjunction with clinical trials;
4. Used in sporting events, having video and consent sent to mobile devices acknowledging the dangers (baseball-balls-bats); Hockey-Pucks; Auto racing events (the delivery could be delivered to specific seating areas, and not to entire crowd);
5. Used in the contracting process i.e. federal contracts listing the rules and regulations and having express consent as understanding;
6. Use in academia (delivering video content and a test instead of a consent);
7. Reconfigured to deliver specialty menu items and an order form;
8. Shuffled—Have a consent delivered first, then followed by (VIDEO/CONTENT) such as explicit content in higher education (avoid litigation over content).

The tablet would be used as the focal point for initiating and encouraging the patient/physician communication.
1. The front office medical staff who would enter the patients name, and patient number, and TruConsent application loads the procedure specific consent and attaches a video and educational material.
2. The program provides patients with an electronic tablet that has been loaded with shared decision-making module(s) specifically based upon both the diagnosis and the procedure/treatment recommended by the referring physician, and being considered as a treatment choice by the patient.
3. The invention application draws a video of the planned procedure-treatment from the TruConsent modality database. This video is loaded for the patient to view and then it is immediately followed by a series of questions that determine the patient's level of understanding of the prescribed procedure/treatment (CONSENT FORM).
4. After each question, the patient indicates that he/she understands the information by answering “YES.” If the patient indicates that he/she did not understand the statement (“NO” answer), the information is reviewed by the physician or qualified personnel (staff) for further explanation directly with the patient.
5. A KEY POINT-EACH QUESTION MAY BE LINKED BACK TO THE VIDEO or OTHER EDUCATION RESOURCE SO THE PATIENT COULD BE DIRECTED TO RE-VIEW A PARTICULAR PORTION OF THE VIDEO. (Or review the question with office personnel in order to clarify the question).
6. If he/she still was still unsure or confused after the review, the “NO” answer(s) are reviewed by clinical staff as detailed above.
7. Only upon answering yes, affirming that he/she fully understood and agrees to the information.
8. FOR ALL QUESTIONS, is the patient then able to digitally sign the form.
9. Upon signing a picture of the patient is taken and embedded into the record. This electronically signed form, and record of the video watched, is then saved in the patients file and an electronic copy is transmitted for verification that:
   a. the patient received the appropriate education on a certain date,
   b. the patient identified by picture embedded into the record was the one being educated and answering the question,
   c. signature on the form is that of the person ID'd, and
   d. physician staff personnel, also e-sign which is embedded in the document as well.
10. Lastly, through patient education the patient's right of self-decision can be effectively exercised through the delivery of information on point that enables an informed choice. This informed choice then affords the patient the ability to make his or her own determination about treatment.

Additionally, this application has multiple applications outside of healthcare. Specifically, it could be:
1. Used in professional sporting events, having video and consent sent to mobile devices acknowledging the dangers (baseball-balls-bats); Hockey-Pucks; Auto racing events. The delivery could be delivered to specific seating areas, and not to entire crowd. They would have the paying guests, watch video and then sign acknowledging and accepting risks.
2. Used in youth sports (High school, Little league, Softball, Pop Warner-Football) where the educational intro video can be customized to individual leagues, schools, school districts.
3. Used in the contracting process i.e. federal contracts listing the rules and regulations and having express consent as understanding.
4. Use in academia. Delivering video content and a test instead of a consent.
5. Used in special education for content delivered in manner that promotes learning.
6. Used in Legal System (Family Court) A Consent order. The app could deliver relevant legal information video then the consent order which is a financial contract that is voluntarily and jointly agreed by a divorcing or divorced couple to finalize all financial obligations arising from their marriage. A Consent Order is a legally binding financial agreement.

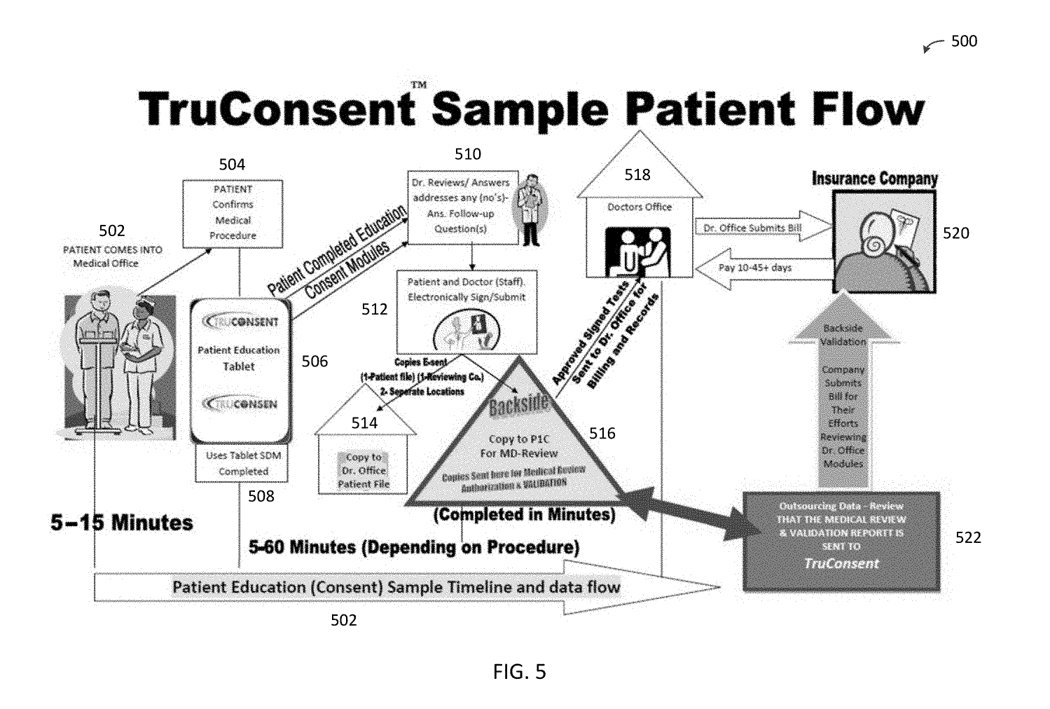

FIG. 5 depicts a sample patient flow 500 in accordance with one embodiment of the present invention. The patient comes into the medical office 500, which starts the patient education (consent) sample timeline and data flow 502. The patient confirms the medical procedure 504 and is given a TruConsent patient education tablet 504 and uses the tablet to complete the SDM 508, which can take 5 to 15 minutes in this example. The doctor reviews the patient's answers, addresses any NOs and answers any follow up questions 510. The patient and doctor, or staff, electronically sign and submit the patient completed education consent modules 512. Copies of the consent are electronically sent to the doctor's office for inclusion in the patient's file 514 and to P1C for medical review authorization and validation 516, which is typically completed in minutes. The approved signed tests are sent to the doctor's office for billing and records 518. The consent process can take 5 to 60 minutes depending on the procedure. The doctor's office submits the bill to the insurance company 520, which can be paid within 10 to 40 days. Outsourcing data and review sent to TruConsent 522, which submits a bill to the insurance company 520 for their efforts reviewing the doctor's office consent modules, which provides backside validation.

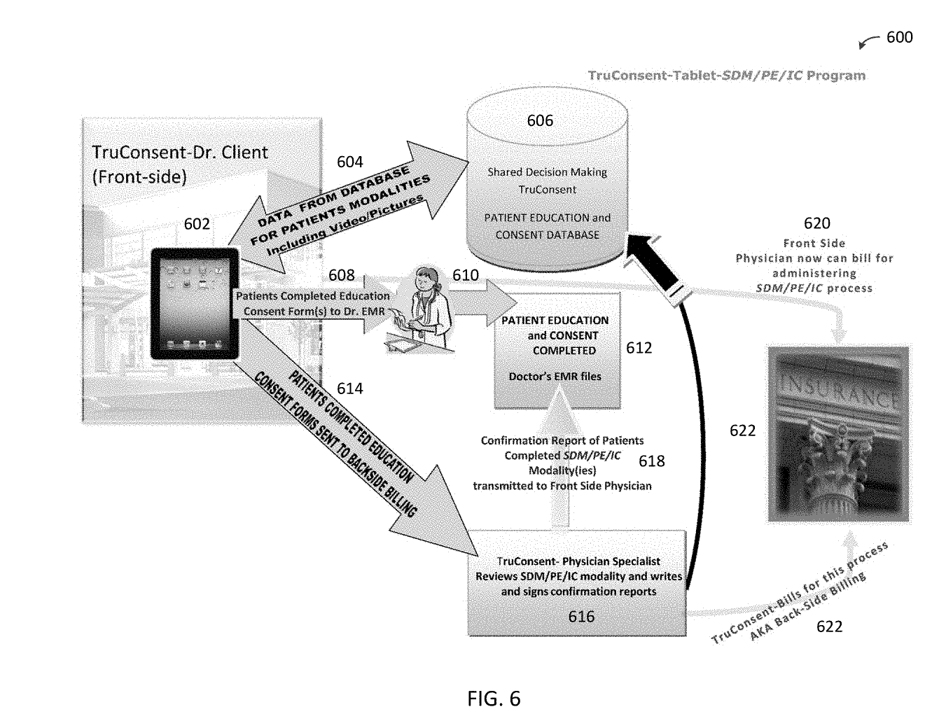

FIG. 6 depicts a data flow 600 in accordance with one embodiment of the present invention. The application runs on a tablet 602 that is provided to the patient and the doctor's office. Alternatively, the application can be executed on the patient's device or computer prior to coming to the doctor's office. Data from the database for the patient's modalities, including videos/pictures, is downloaded 604 into the tablet 602 from the SDM patient education and consent database 606. The patient completes the education/consent forms 608 on the tablet, which are reviewed by the doctor or doctor's staff 610 and provided to the doctor's EMR 612. The patient's completed education consent forms are automatically sent to backside billing 614 via email where a TruConsent physician specialist 616 reviews the SDM/PE/IC modality and writes and signs a confirmation report. TruConsent physician specialist 616 is not a treating physician, and is only performing a education—completeness verification. The report should be a copy of completed consent form and video in which all questions answered yes, the patient's signature and photo (or photo of their ID), and the front side staff's information (signature, etc.). The report must have all patient information included for billing. The confirmation report of the patient's completed SDM/PE/IC modalities 618 is sent (e.g., reviewer hitting link in report body) to the doctor's EMR files 612 and the patient education and consent database 606. The physician can now bill 620 the insurance company or government 622 for administering the SDM/PE/IC process. TruConsent can also bill 624 the insurance company or government 620 for the process.

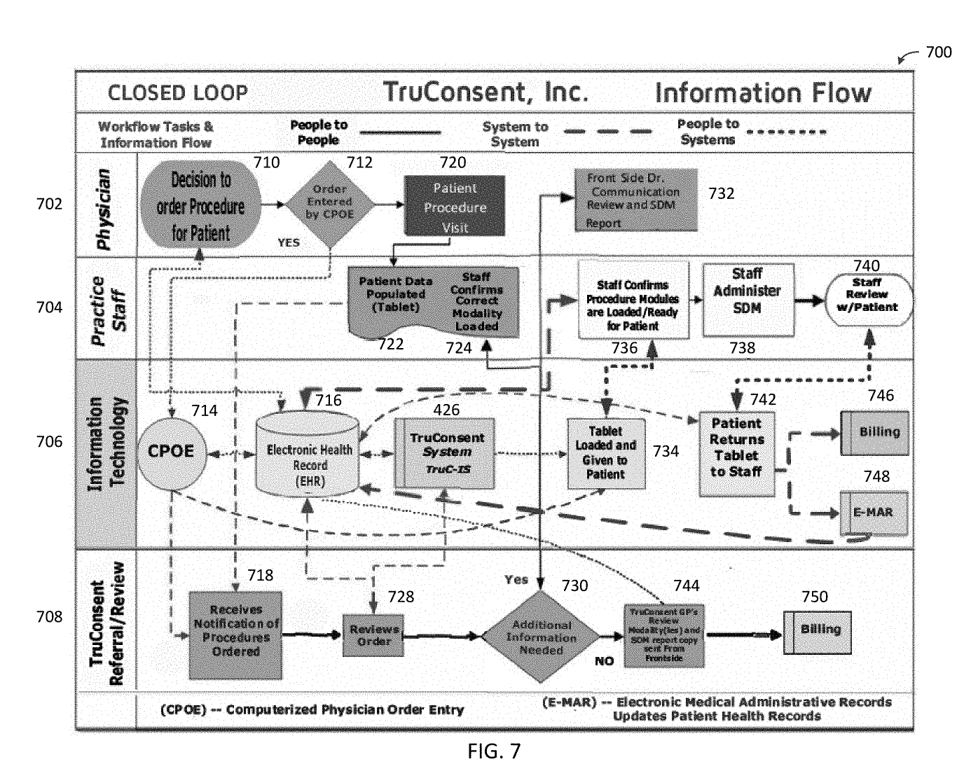

FIG. 7 depicts an information flow 700 in accordance with one embodiment of the present invention. The workflow tasks and information flow are people to people as indicated by solid line, system to system as indicated by long dashed lines, and people to systems as indicated by short dashed lines. In this example, the information flow involves the physician 702, practice staff 704, information technology 706 and TruConsent referral/review 708. The physician 702 decides to order a procedure for the patient in block 710 and if the order is entered by computerized physician order entry (CPOE), as determined in decision block 712, the CPOE 714 is entered into the electronic health record (EHR) 716 and TruConsent 708 receives notification of the procedures ordered 718. During the patient procedure visit 720, the tablet is populated with the patient's data 722 and the staff 704 confirms that the correct modality data is loaded on the tablet 724. The EHR 716 is also provided to the TruConsent system (TruC-IS) 726 and TruConsent 708 reviews the order along with the patient data 728. If additional information is needed, as determined in decision block 730, the physician 702 reviews the information and SDM report 732. The tablet is loaded with the consent modules and made ready 734 to given to the patient. The Staff 704 confirms the procedure modules are loaded on the tablet and ready for the patient 736. The staff 704 administers the SDM 738 and reviews the SDM with the patient 740. The patient returns the tablet 742 to the staff 704. TruConsent's GPs 708 review the modality(ies) and SDM report copy sent from the frontside 744. Billing 746 and electronic medical administrative records updates patient health records (E-MAR) 748 are processed for the doctor's office. TruConsent 708 also bills 750 for the process.

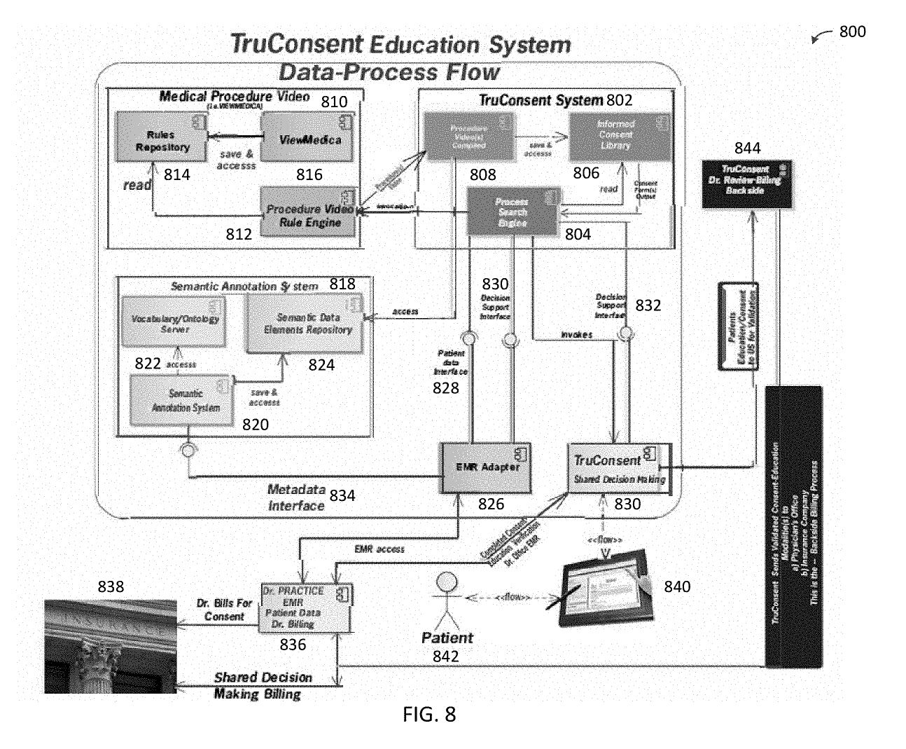

FIG. 8 depicts an education system data-process flow 800 in accordance with one embodiment of the present invention. The TruConsent System 802 includes a process search engine 804 that reads from and receives consent forms(s) from an informed consent library 806, which saves and accesses compiled video(s) procedure 808. The Medical Procedure Video (i.e., ViewMedica, etc.) 810 includes a procedure video rule engine 812 that reads from a rules repository 814, which saves and accesses ViewMedica 816. The process search engine 804 invokes the procedure video rule engine 812. The Semantic Annotation System 818 includes a semantic annotation system 820 that accesses a vocabulary/ontology server 822, and saves and accesses data in a semantic data elements repository 824. The compiled video(s) procedure 808 accesses the semantic data elements repository 824. The process search engine 804 also communicates with an EMR adapter 826 via a patient data interface 828 and a decision support interface 830. The process search engine 804 also invokes the TruConsent shared decision making 830 and includes a decision support interface 832. The EMR adapter 826 has a metadata interface 834 to the semantic annotation system 820, and accesses the doctor's EMR patient data and billing 836, which bills the insurance company or government 838 for the consents. The TruConsent shared decision making 830 interfaces with the tablet 840, which is provided to the patient 842. The TruConsent shared decision making 830 also receives the completed verification by the doctor's EMR 836 and provides the patient's education/consent to the TruConsent doctor review-billing backside 844, which sends the validated consent-education modalities to the physician's office EMR 836 and insurance company or government 838.

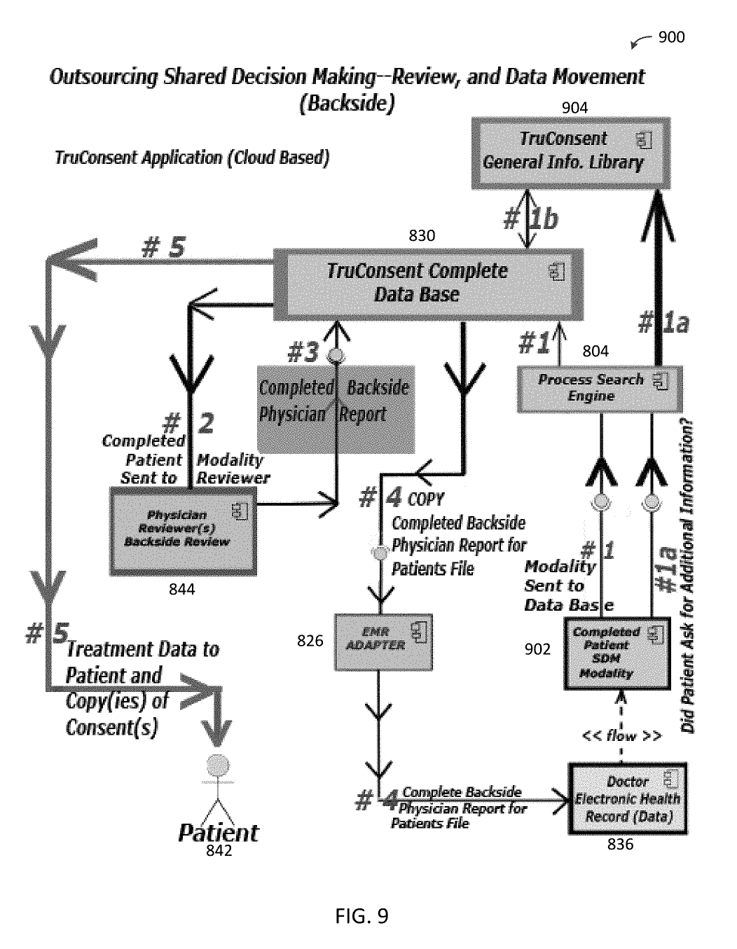

FIG. 9 depicts an outsourcing shared decision making, review and data movement flow (backside) 900 in accordance with one embodiment of the present invention. The doctor's electronic health record (data) 836 for the patient is used to create the completed patient SDM modality 902, which is sent (#1) to the TruConsent complete database 830 via process search engine 804. If the patient asked for additional information (#1a), the additional information is obtained from the TruConsent general information library 904 via process search engine 804 (#1a) and provided (#1b) to the TruConsent complete database 830. The completed patient modality (#2) is sent from the TruConsent complete database 830 to the physician reviewer(s) 844 for backside review. The completed backside physician report (#3) is sent to the TruConsent complete database 830. A copy of the completed backside physician report (#4) for the patient's file is sent from the TruConsent complete database 830 to the EMR adapter 826, which is then saved (#4) in the doctor's electronic health record 836. The TruConsent complete database 830 sends the treatment data and copy(ies) of consent(s) (#5) to the patient 842.

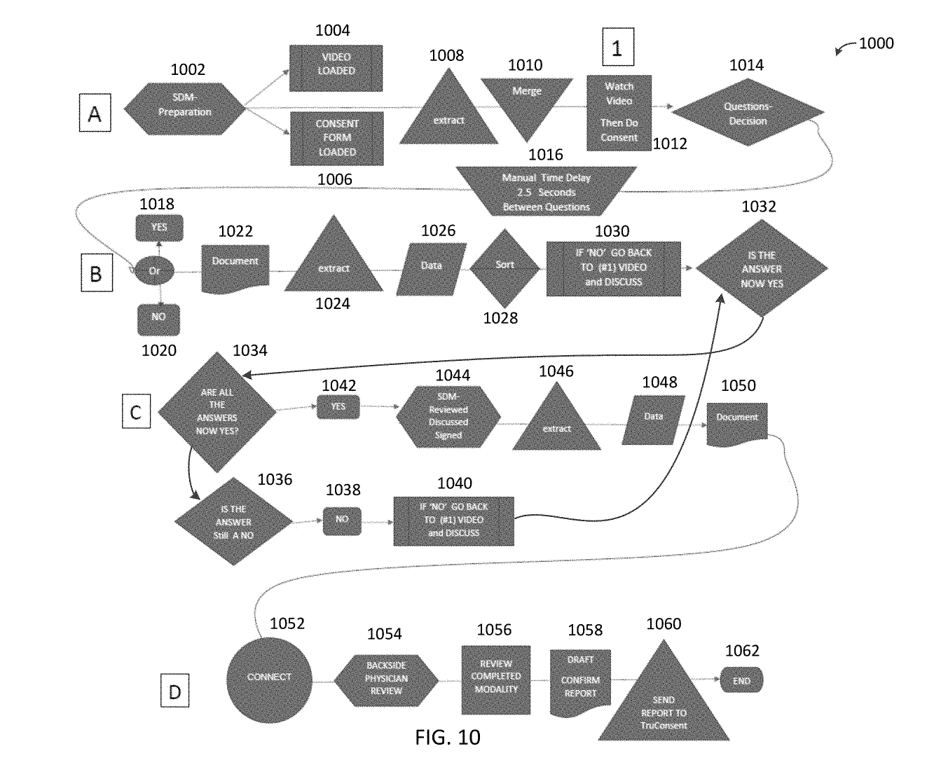

FIG. 10 depicts a process flow 1000 in accordance with one embodiment of the present invention. The process 1000 involve four basic section revolving around providing and obtaining the consent [1], namely preparation [A], question-answers [B], doctor/staff review [C] and TruConsent review [D]. The SDM preparation begins at block 1002 in which videos 1004 and consent forms 1006 are loaded. The education/consent is extracted 1008 and merged 1010, and presented to the patient. The patient watches the video and stars the consent 1013, which is a series of questions and/or decisions 1014 that require a YES 1018 or NO 1020 response. The responses are documented 1022, extracted 1024 and the data 1026 is sorted 1028. If there are any NOs 1030, the process goes back to [1] for the videos and discussion with the doctor or staff. If the answer is now YES 1032, but there is still a NO 1038 answer as determined in block 1036, the process 1040 goes back to [1] for the videos and discussion with the doctor or staff. If all the answers are YES 1042, as determined in decision block 1034, the SDM is reviewed, discussed and signed by the doctor or staff 1044. The SDM is extracted 1046 and the data 1048 loaded into a document 1050. A connection 1052 is made to TruConsent for backside physician review 1054 of the document. The modality review is completed 1056 and the report is drafted and confirmed 1058.

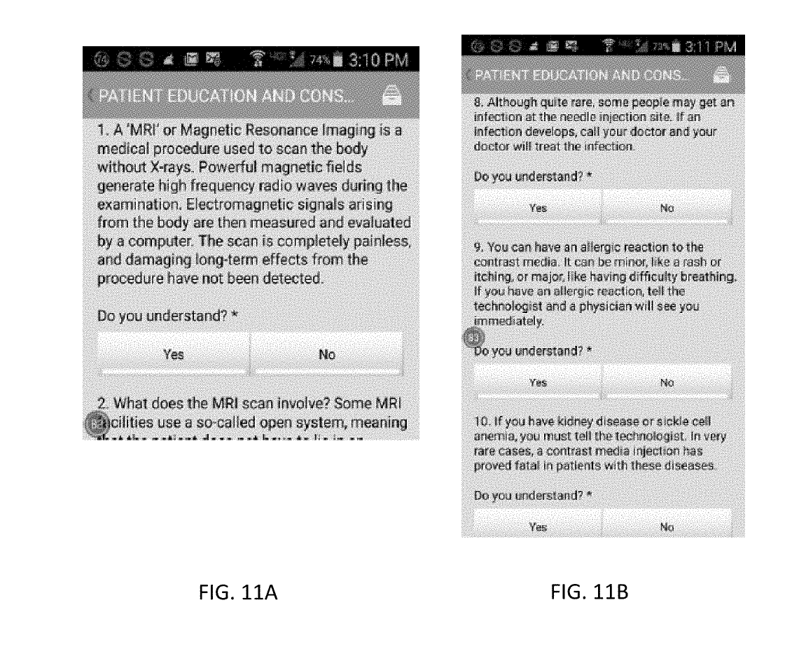

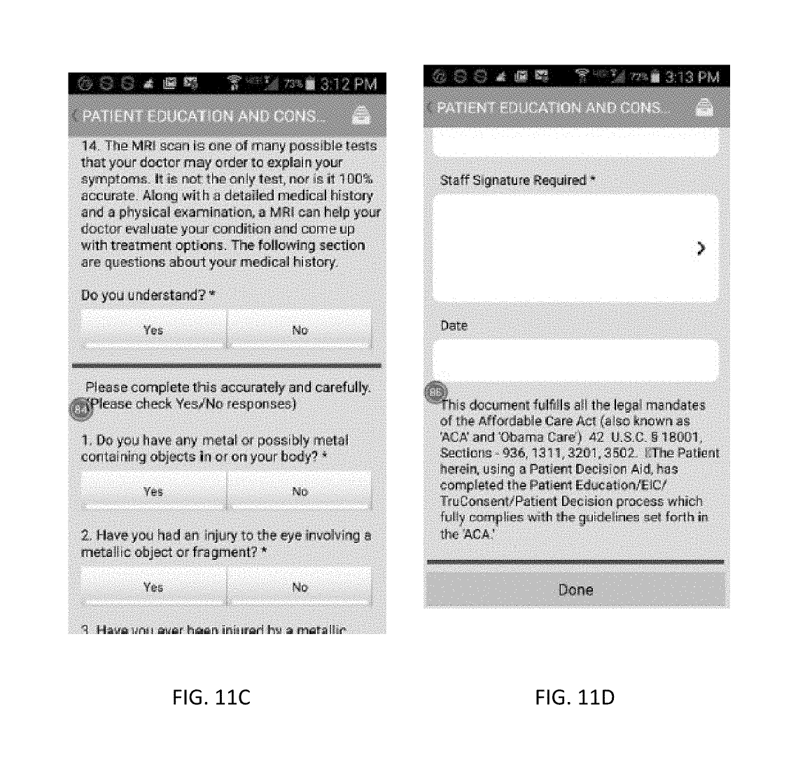

FIGS. 11A-11D are screen shots of yes/no questions presented on a device to a patient for a MRI scan in accordance with one embodiment of the present invention.

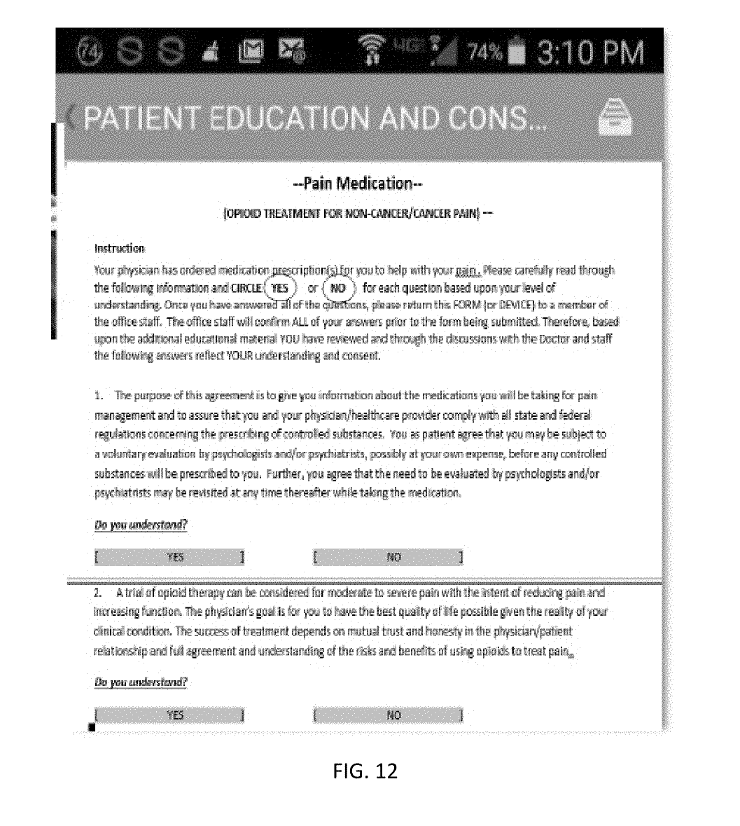

FIG. 12 is a screen shot of yes/no questions presented on a device to a patient for a pain medication in accordance with one embodiment of the present invention.

As illustrated by the non-limiting examples described above, anntegrated system, program-method to supply healthcare community with an outsourced Shared Decision-Making resources using a hybrid computing environment comprising standalone, client-server, or Internet controlling private healthcare information for shared patient and physician decision making is presented here. The application can be installed on a client computer that is configured for use as either a standalone or networked computer. Server components of the application program are installed on a server computer configured to be coupled to the client computer over a peer-to-peer network, client-server network or a large-scale network, such as the Internet. Data and file resources utilized by the application program are accessed from the server computer, coupled to the network coupling the client and server computers. Connectivity between the client and server computers and interoperability of the application program components is through an integrated application to facilitate the sharing of information between the healthcare provider network and the application.

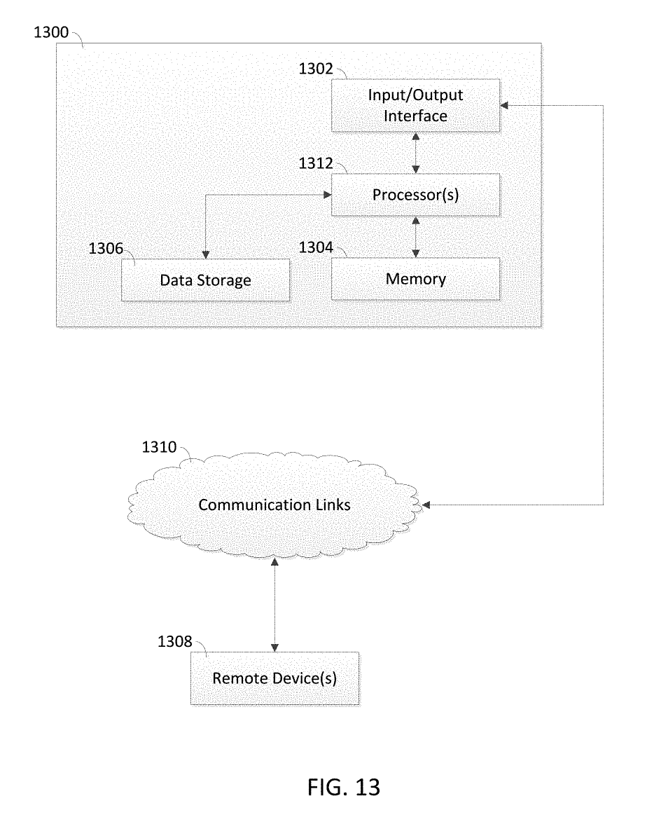

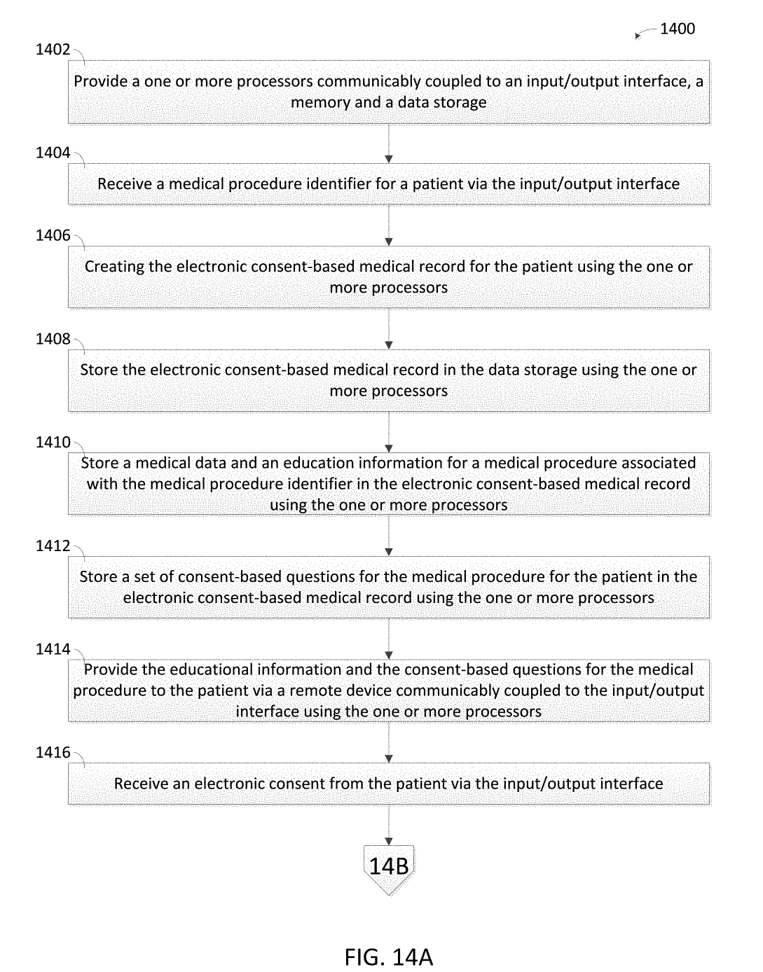

FIG. 13 depicts a system 1300 for creating an electronic consent-based medical record in accordance with one embodiment of the present invention. The system 1300 includes an input/output interface 1302, a memory 1304, a data storage 1306, a remote device 1308 communicably coupled to the input/output interface 1302 via one or more communication links 1310, and one or more processors 1312 communicably coupled to the input/output interface 1302, the memory 1304, and the data storage 1306. The one or more processors 1312 receive a medical procedure identifier for a patient via the input/output interface 1302, create the electronic consent-based medical record for the patient and store the electronic consent-based medical record in the data storage 1306, store a medical data and an education information for a medical procedure associated with the medical procedure identifier in the electronic consent-based medical record, store a set of consent-based questions for the medical procedure for the patient in the electronic consent-based medical record, provide the educational information and the consent-based questions for the medical procedure to the patient via the remote device 1308, receive an electronic consent from the patient via the input/output interface 1302, store the electronic consent in the electronic consent-based medical record, provide a report based on the electronic consent-based medical record to a medical provider via the input/output interface 1302, receive an electronic authorization from the medical provider via the input/output interface 1302, and store the electronic authorization in the electronic consent-based medical record.

In one aspect, the one or more processors obtain the medical data for the patient, the educational information for the medical procedure or a combination thereof. In another aspect, the one or more processors obtain or generate the set of consent-based questions for the medical procedure associated with the medical procedure identifier for the patient. In another aspect, the one or more processors create an executable package or file that provides the education information or the consent-based questions or both to the patient when activated by the remote device, and wherein providing the educational information and the consent-based questions comprises sending the executable package or file to the remote device via the input/output interface. In another aspect, the consent-based questions comprise an automated question-answer session. In another aspect, the one or more processors request the electronic consent from the patient after a successful completion of the consent-based questions. In another aspect, the electronic consent comprises an electronic signature, a biometric identifier, an image of the patient taken by the remote device, or a combination thereof. In another aspect, the one or more processors: receive a request for an additional information regarding the medical procedure from the remote device; and provide the additional information to the remote device or notifying the medical provider of the request. In another aspect, the one or more processors generate the report based on the electronic consent-based medical record. In another aspect, the one or more processors update a patient health record based on the electronic consent-based medical record. In another aspect, the one or more processors generate a bill or medical reimbursement request based on the electronic consent-based medical record. In another aspect, the one or more processors: provide the educational information and/or the consent-based questions to the medical provider prior to providing the educational information and the consent-based questions to the patient; receive an authorization to provide the consent-based question to the patient; and store the authorization in the electronic consent-based medical record. In another aspect, the one or more processors provide a patient report based on the electronic consent-based electronic record. In another aspect, the educational information comprises one or more of a video, an audio recording, an electronic document, an electronic presentation, an image, or a hyperlink. In another aspect, all communications via the input/output interface are encrypted.

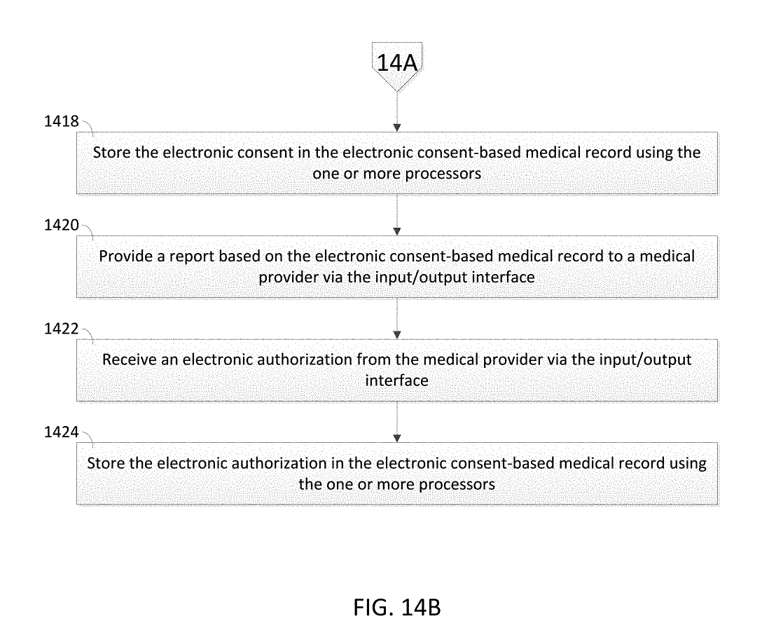

FIGS. 14A-B depict a computerized method 1400 for creating an electronic consent-based medical record in accordance with one embodiment of the present invention. One or more processors communicably coupled to an input/output interface, a memory and a data storage are provided in block 1402. A medical procedure identifier for a patient is received via the input/output interface in block 1404. The electronic consent-based medical record for the patient is created in block 1406, and the electronic consent-based medical record is stored in the data storage in block 1408 using the one or more processors. A medical data and an education information for a medical procedure associated with the medical procedure identifier is stored in the electronic consent-based medical record using the one or more processors in block 1410. A set of consent-based questions for the medical procedure for the patient is stored in the electronic consent-based medical record using the one or more processors in block 1412. The educational information and the consent-based questions for the medical procedure are provided to the patient via a remote device communicably coupled to the input/output interface using the one or more processors in block 1414. An electronic consent is received from the patient via the input/output interface in block 1416. The electronic consent is stored in the electronic consent-based medical record using the one or more processors in block 1418. A report based on the electronic consent-based medical record is proved to a medical provider via the input/output interface in block 1420. An electronic authorization is received from the medical provider via the input/output interface in block 1422. The electronic authorization is stored in the electronic consent-based medical record using the one or more processors in block 1424.

In one aspect, the method further comprises obtaining the medical data for the patient, the educational information for the medical procedure or a combination thereof. In another aspect, the method further comprises obtaining or generating the set of consent-based questions for the medical procedure associated with the medical procedure identifier for the patient. In another aspect, the method further comprises creating an executable package or file using the one or more processors that provides the education information or the consent-based questions or both to the patient when activated by the remote device, and wherein providing the educational information and the consent-based questions comprises sending the executable package or file to the remote device via the input/output interface. In another aspect, the consent-based questions comprise an automated question-answer session. In another aspect, the method further comprises requesting the electronic consent from the patient after a successful completion of the consent-based questions. In another aspect, the electronic consent comprises an electronic signature, a biometric identifier, an image of the patient taken by the remote device, or a combination thereof. In another aspect, the method further comprises receiving a request for an additional information regarding the medical procedure from the remote device; and providing the additional information to the remote device or notifying the medical provider of the request. In another aspect, the method further comprises generating the report based on the electronic consent-based medical record. In another aspect, the method further comprises updating a patient health record based on the electronic consent-based medical record. In another aspect, the method further comprises generating a bill or medical reimbursement request based on the electronic consent-based medical record. In another aspect, the method further comprises providing the educational information and/or the consent-based questions to the medical provider prior to providing the educational information and the consent-based questions to the patient; receiving an authorization to provide the consent-based question to the patient; and storing the authorization in the electronic consent-based medical record. In another aspect, the method further comprises providing a patient report based on the electronic consent-based electronic record. In another aspect, the educational information comprises one or more of a video, an audio recording, an electronic document, an electronic presentation, an image, or a hyperlink. In another aspect, all communications via the input/output interface are encrypted.

Note that the foregoing method and any of the aspects can be implemented using a non-transitory computer readable medium that when executed by the one or more processors performs the stated functionality.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above”, “below”, “upper”, “lower”, or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

1. A computerized method for creating an electronic consent-based medical record comprising:
providing one or more processors communicably coupled to an input/output interface, a memory and a data storage;
receiving a medical procedure identifier for a patient via the input/output interface;
creating the electronic consent-based medical record for the patient and storing the electronic consent-based medical record in the data storage using the one or more processors;
storing a medical data and an education information for a medical procedure associated with the medical procedure identifier in the electronic consent-based medical record using the one or more processors;
storing a set of consent-based questions for the medical procedure for the patient in the electronic consent-based medical record using the one or more processors;
providing the educational information and the consent-based questions for the medical procedure to the patient via a remote device communicably coupled to the input/output interface using the one or more processors;
receiving an electronic consent from the patient via the input/output interface;
storing the electronic consent in the electronic consent-based medical record using the one or more processors;
providing a report based on the electronic consent-based medical record to a medical provider via the input/output interface;
receiving an electronic authorization from the medical provider via the input/output interface; and
storing the electronic authorization in the electronic consent-based medical record using the one or more processors.

2. The method of claim 1, further comprising obtaining the medical data for the patient, the educational information for the medical procedure or a combination thereof.

3. The method of claim 1, further comprising obtaining or generating the set of consent-based questions for the medical procedure associated with the medical procedure identifier for the patient.

4. The method of claim 1, further comprising creating an executable package or file using the one or more processors that provides the education information or the consent-based questions or both to the patient when activated by the remote device, and wherein providing the educational information and the consent-based questions comprises sending the executable package or file to the remote device via the input/output interface.

5. The method of claim 1, wherein the consent-based questions comprise an automated question-answer session.

6. The method of claim 1, further comprising requesting the electronic consent from the patient after a successful completion of the consent-based questions.

7. The method of claim 1, wherein the electronic consent comprises an electronic signature, a biometric identifier, an image of the patient taken by the remote device, or a combination thereof.

8. The method of claim 1, further comprising:
receiving a request for an additional information regarding the medical procedure from the remote device; and
providing the additional information to the remote device or notifying the medical provider of the request.

9. The method of claim 1, further comprising generating the report based on the electronic consent-based medical record.

10. The method of claim 1, further comprising updating a patient health record based on the electronic consent-based medical record.

11. The method of claim 1, further comprising generating a bill or medical reimbursement request based on the electronic consent-based medical record.

12. The method of claim 1, further comprising:
providing the educational information and/or the consent-based questions to the medical provider prior to providing the educational information and the consent-based questions to the patient;
receiving an authorization to provide the consent-based question to the patient; and
storing the authorization in the electronic consent-based medical record.

13. The method of claim 1, further comprising providing a patient report based on the electronic consent-based electronic record.

14. The method of claim 1, wherein the educational information comprises one or more of a video, an audio recording, an electronic document, an electronic presentation, an image, or a hyperlink.

15. The method of claim 1, wherein all communications via the input/output interface are encrypted.

16. A system for creating an electronic consent-based medical record comprising:
an input/output interface;
a memory;
a data storage;
a remote device communicably coupled to the input/output interface;
one or more processors communicably coupled to the input/output interface, the memory and the data storage, wherein the one or more processors receive a medical procedure identifier for a patient via the input/output interface, create the electronic consent-based medical record for the patient and store the electronic consent-based medical record in the data storage, store a medical data and an education information for a medical procedure associated with the medical procedure identifier in the electronic consent-based medical record, store a set of consent-based questions for the medical procedure for the patient in the electronic consent-based medical record, provide the educational information and the consent-based questions for the medical procedure to the patient via the remote device, receive an electronic consent from the patient via the input/output interface, store the electronic consent in the electronic consent-based medical record, provide a report based on the electronic consent-based medical record to a medical provider via the input/output interface, receive an electronic authorization from the medical provider via the input/output interface, and store the electronic authorization in the electronic consent-based medical record.

17. The system of claim 16, wherein the one or more processors obtain the medical data for the patient, the educational information for the medical procedure or a combination thereof.

18. The system of claim 16, wherein the one or more processors obtain or generate the set of consent-based questions for the medical procedure associated with the medical procedure identifier for the patient.

19. The system of claim 16, wherein the one or more processors create an executable package or file that provides the education information or the consent-based questions or both to the patient when activated by the remote device, and wherein providing the educational information and the consent-based questions comprises sending the executable package or file to the remote device via the input/output interface.

20. The system of claim 16, wherein the consent-based questions comprise an automated question-answer session.

21. The system of claim 16, wherein the one or more processors request the electronic consent from the patient after a successful completion of the consent-based questions.

22. The system of claim 16, wherein the electronic consent comprises an electronic signature, a biometric identifier, an image of the patient taken by the remote device, or a combination thereof.

23. The system of claim 16, wherein the one or more processors:
receive a request for an additional information regarding the medical procedure from the remote device; and
provide the additional information to the remote device or notifying the medical provider of the request.

24. The system of claim 16, wherein the one or more processors generate the report based on the electronic consent-based medical record.

25. The system of claim 16, wherein the one or more processors update a patient health record based on the electronic consent-based medical record.

26. The system of claim 16, wherein the one or more processors generate a bill or medical reimbursement request based on the electronic consent-based medical record.

27. The system of claim 16, wherein the one or more processors:
provide the educational information and/or the consent-based questions to the medical provider prior to providing the educational information and the consent-based questions to the patient;
receive an authorization to provide the consent-based question to the patient; and
store the authorization in the electronic consent-based medical record.

28. The system of claim 16, wherein the one or more processors provide a patient report based on the electronic consent-based electronic record.

29. The system of claim 16, wherein the educational information comprises one or more of a video, an audio recording, an electronic document, an electronic presentation, an image, or a hyperlink.

30. The system of claim 16, wherein all communications via the input/output interface are encrypted.
